(19) United States
(12) Patent Application Publication
(10) Pub. No.: US 2006/0016054 A1
(43) Pub. Date: Jan. 26, 2006

(54) Light duty consumable prosthesis

(76) Inventor: Sean A. Muzzillo, Phoenix, AZ (US)

Correspondence Address: LAW OFFICES OF DOUGLAS W RUDY LLC;14614 NORTH KIERLAND BLVD, SUITE 300, SCOTTSDALE, AZ, 85254, US

(21) Appl. No.: 10/895,008

(22) Filed: Jul. 20, 2004

(51) Int. Cl. A61F 2/28

(52) U.S. Cl. 27/21.1; 623/23.47

(57) ABSTRACT

A light duty consumable prosthesis for use in replicating leg bones, arm bones and spinal column vertebra in a cadaver is constructed of largely consumable products to enable the prosthesis to be incinerated in the event that the host cadaver is cremated. The leg prosthesis is telescoping such that it is length adjustable to be sizable in cadavers of different leg lengths. The arm prosthesis is also of a telescoping design that includes a bendable hinge between an upper arm portion and a lower arm portion. The spinal column prosthesis is similar to the upper arm prosthesis and includes rods to assist in securing it in location.

### BACKGROUND OF THE INVENTION

This invention is a light duty consumable prosthesis for use in cadavers to take the place of bones that have been removed from cadavers for medical and/or scientific purposes. Various organizations, such as tissue banks, hospitals, and organ donor associations may surgically remove larger bones from cadavers for scientific and medical reasons such as for donor-to-recipient transplants and tissue research. A replacement for the removed bone component is desirable to preserve visual and structural integrity of the cadaver. The device presented is a light duty prosthesis simulating a bone section. The prosthesis may be placed and positioned in a cadaver to provide tissue support and structure to a limb from which the natural bone has been removed.

### SUMMARY OF THE INVENTION

An extendable apparatus used as a prosthesis to replace a bone segment in a cadaver is provided. Extension and telescoping capability allow the prosthesis to be extended to a required length to fit into a recipient cavity. The apparatus includes a locking device to prevent telescoping after the locking mechanism has been activated. The light duty consumable prosthesis is designed to be reduced to a minimal amount of insignificant residue when incinerated in a crematory facility.

One problem with a prosthesis used in cadavers is that the prosthesis may be made of a plastic material, such as polyvinyl chloride, that is not consumed in a cremation situation. Cremation of a cadaver is sometimes the chosen method of dealing with a donor body after it has been processed. Plastic prosthesis elements will melt, however the plastic material will not be totally consumed in the cremation process. The prosthesis presented here is made of consumable stock that is acceptable to receiving crematoriums.

It is known to provide a prosthesis to replace bones in a donor cadaver. One embodiment comprises a wooden rod that is cut to an appropriate length to replace the bone that has been removed. This crude prosthesis has technologically evolved into length adjustable units of wood or plastic. One wooden device is made of two compatible sliding pieces, each of the two wooden components having one or more holes drilled therein to ultimately line up with a hole in the mating component. The two wooden components slide relative to each other to attain a desired length and are then pinned together, by a “pin-in-hole” register system, by inserting a pin through aligned ones of the aforesaid holes.

A second type of prosthesis uses plastic pipe to form the structural elements. Two tubes or pipes are used with one tube fitting inside a second tube. One tube has an inside diameter slightly larger then the outside diameter of the other tube thus fitting inside the larger diameter tube. The tubes are provided with one or more holes that will allow pin passage for an indexing pin to pass through and lock the two tube components together at the selected length for the prosthesis.

### BRIEF DESCRIPTION OF THE DRAWINGS

Various embodiments of the present invention are described below with reference to the drawings figures, wherein like designations denote like elements.

FIG. 1 is a pictorial representation of the prosthesis of this invnetion in an unextended state.

FIG. 2 is a pictorial representation of the prosthesis of FIG. 1 in an extended state.

FIG. 3 is a portion of a dowel component of the prosthesis of FIGS. 1 and 2 showing the cam restraint element at one end of the dowel.

FIG. 4 is a top view of the cam restraint element shown in FIG. 3.

FIG. 5 is a prosthesis for use as an arm prosthesis including a bendable joint.

FIG. 6 is the prosthesis of FIG. 5 showing various elements extended and displaced from the position shown in FIG. 5.

FIG. 7 is an upper arm, adjustable length prosthesis in an unextended state.

FIG. 8 is an extendable prosthesis used as a simulated section of a spinal column.

### DESCRIPTION OF PREFERRED EXEMPLARY EMBODIMENTS

While the invention is described herein in terms of preferred embodiments, the inventor contemplates that alterations and permutations of the preferred embodiments will become apparent to those skilled in the art upon a reading of the specification and a study of the drawing figures. Below is a description of variations of prosthesis embodiments contemplated by the inventor and falling within the scope of the claims unless excluded by specific claim language.

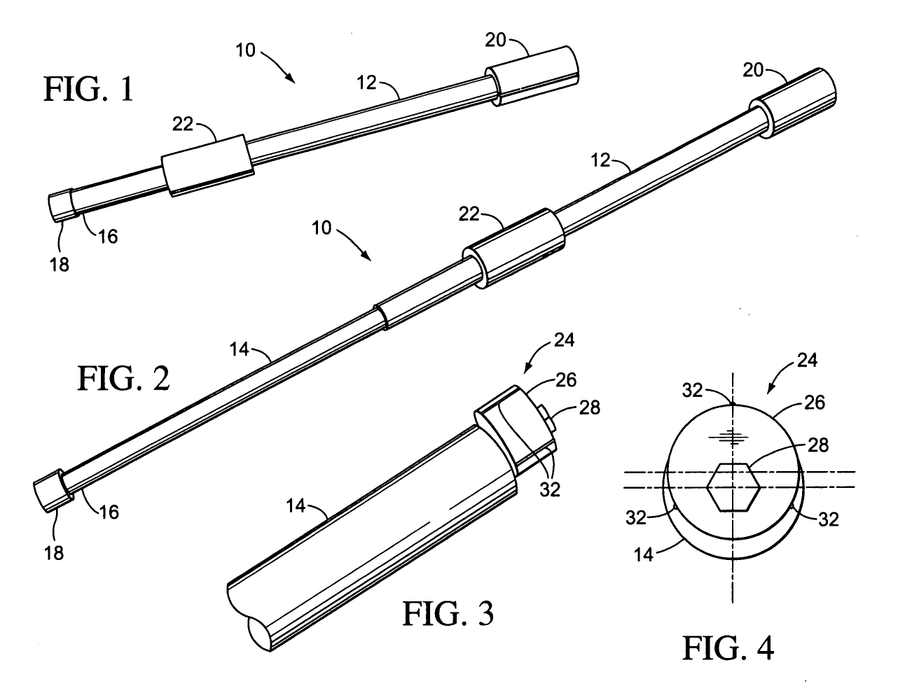

The invention will be understood by a perusal of the drawing figures as described in the following description and enablement of the invention. Starting with FIGS. 1 and 2, a leg prosthesis, generally 10, is shown. The leg prosthesis, the arm prosthesis and the spine prosthesis described further on, is designed to simulate, not to exactly replicate, the bone structure in a human leg, a human arm and a spine respectively. Therefore actual bone nomenclature, such as femur, tibia, patella, fibula, and humerus, isn't used in this description.

A tubular element 12, in a preferred embodiment, a cardboard tube which may have a fluid resistant external surface, and a generally hollow interior is provided. The tube 12 is approximately 2 inches in external diameter and has a nominal inside diameter of approximately 1.5 inches. The size of the tube is not critical to the invention and the aforesaid dimensions are presented as examples only. A dowel 14, has an outside diameter closely matched to the inside diameter of the tube 12. The dowel 14 may be a wooden dowel in a preferred embodiment but may be made of other consumable materials such as cardboard or a cardboard tube or the like. Thus, in one example, the outside diameter of the wooden dowel 14 is nominally 1.5 inches. The wooden dowel 14 will be a slidable fit inside the tube 12. The outboard end 16 of the wooden dowel 14 may be provided with a enlarged end element 18. This enlarged end element 18 may have an outside diameter similar to the diameter of the tube 12 and an inside diameter of the tube 12 as well. In one embodiment the enlarged element 18 is a section of tubing cut from the same tubing stock used for the tube 12. As the prosthesis, generally 10, is a leg prosthesis, the enlarged element is provided to simulate an ankle joint.

In FIGS. 1 and 2 a first hip emulating element 20 is shown. This element may be made of a firm foam extruded tube, or the like, that is positioned around the exterior diameter of the tubular element 12. It is provided to simulate the hip of the recipient of the prosthesis. A second foam element 22, a knee simulating element, is also positioned on the tubular element 12. This second foam element 22 is generally slidable along the length of the tube 12 such that it is positioned to approximate the knee location relative to the hip and ankle location of the recipient cadaver.

FIGS. 1 and 2 illustrate that the dowel 14, here a wooden dowel or the like, is slidably carried in the interior of the tube 12 and that the pseudo-knee element 22 is slidable on the exterior of the tube 12. The hip emulating element 20 is fixed to the tube 12 in one contemplated embodiment. The hip emulating element 20 may be slidably positionable as an alternative to being fixed in location. The ankle simulating element 18 is generally fixed to the end of the dowel 14.

FIGS. 3 and 4 present the apparatus used to stabilize the lateral position of the wooden dowel 14 in the tube 12. An eccentric cam locking device, generally 24, is used for this purpose. The eccentric cam device 24 includes a cylindrical block 26 having a diameter slightly smaller than the outside diameter of the wooden dowel 14. The cylindrical block 26 is provided with a through bore (not shown) that is formed in the cylindrical block offset from the diametric center of the block. A fastener, serving as an axle 28, is positioned through the through bore of the cylindrical block 26. This fastener 28, in one embodiment it may be a threaded screw as shown, is then threaded into the end of the wooden dowel 14. The end of the wooden dowel 14 may generally be provided with a centrally drilled pilot hole into which the axle 28 is threaded or otherwise fitted and carried. The wooden dowel 14, or the like, having an outside diameter closely fit the internal diameter of the tube 12 such that there is a press fit between the tube 12 and the wooden dowel 14.

A feature of the cylindrical block 26 is the provision of a protrusion or preferably several protrusions such as, but not limited to, the external longitudinal ribs 32 formed on the exterior surface of the cylindrical block 26. These longitudinal ribs 32 provide friction elements between the cylindrical block 26 and the interior of the tube 12 so that when the wooden dowel 14 is inserted into the tube 12, positioned laterally to a desired length and then rotated, the cam locking elements 24 will cause friction interference between the tube 12 and the wooden dowel 14 such that the relative longitudinal positions of the tube and the wooden dowel 14 will be fixed due to the pressure of the surface of the wooden dowel 14 and the interior surface of the tube 12.

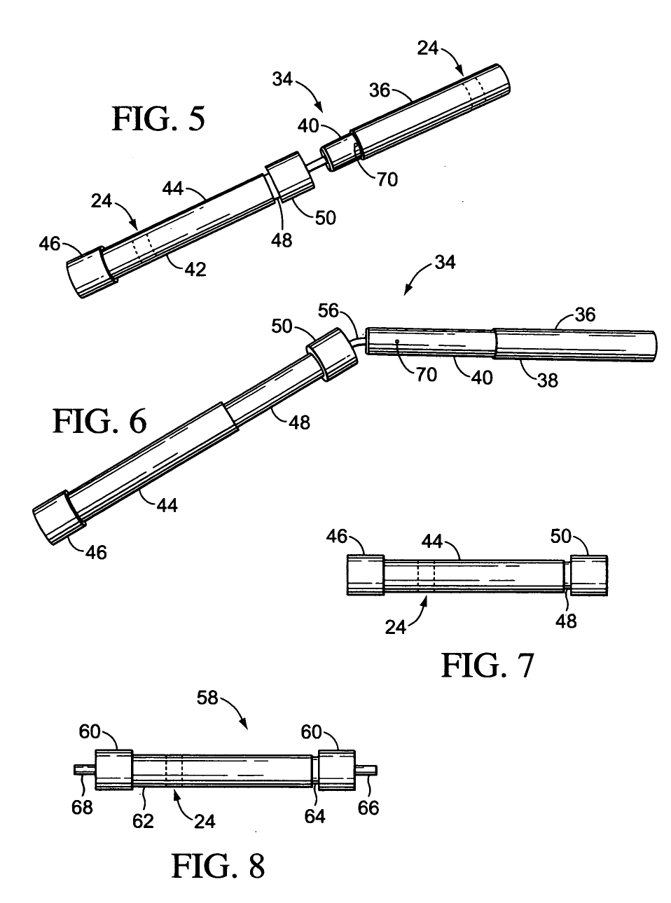

Before looking at FIGS. 5 and 6 the operation of the leg prosthesis shown in FIGS. 1-4 will be further explained. The leg prosthesis will replace leg bones removed from a cadaver. Such bones could have been removed to replace defective bone in a recipient or used for scientific or medical research. The donor cadaver will have a cavity resulting from the removal of its bone structure. The leg prosthesis will be placed in such cavity and adjusted for length and knee (second foam element) position. The knee component, the second foam element 22, is slid along the tubular element 12 toward the end of the tube 12 away from the hip emulating element 20 into a position approximating the natural position of the cadaver's knee. With the knee element 22 positioned, the length of the leg prosthesis can be adjusted. The wooden dowel 14 will be telescopically extended from the interior of the tubular element 12 so that the ankle 18, the enlarged end element, is positioned approximating the normal position of the ankle of the cadaver. When the positioning, primarily settling on the length of the leg of the prosthesis, is in the desired position, the wooden dowel 14, is rotated along its major axis while the tubular element 12 is held steady. The rotation of the wooden dowel 14 will cause the cylindrical block to contact the interior of the tube and, due to its eccentric association with the dowel, will cause firm contact between the dowel and the tube thus restricting relative movement of the tube 12 and the dowel 14. This is accomplished by the eccentric cam locking device, generally 24, rotating into a locking position such that at least a portion of exterior surface of the wooden dowel 14 is forced against the interior surface of the tube 12, generally opposite the longer diametric vector of the cylindrical block 26. The eccentric cam locking device 24 provides a significant improvement over the pin-into-hole length adjustment prosthesis known to the inventor in that the tube and the dowel of this invention don't have to be rotated and aligned to get a pair of holes lined up to accept a pin of the pin-into-hole type devices. Also the instant invention provides infinite adjustability of the length of the leg bone prosthesis while pin-in-hole length registration devices have a limited incremental length selection.

The leg bone prosthesis, once positioned, in the cadaver can remain with the cadaver through the cadaver's transportation and final disposition whether the cadaver be buried or cremated. The leg prosthesis is consumable to a large extent in the cremation process. The cardboard tube 12 and the wooden dowel 14 are fully consumable. The ankle joint is fully consumable and the second foam element 22, the simulated knee joint, and the hip emulating element 20, the foam element 20, are virtually totally consumed as they are low density products that will be reduced to insignificant ash. The cylindrical block 26, a plastic material in one embodiment, but alternatively a block made of more consumable material, presents an insignificant quantity of material upon cremation, that has been shown to be unobjectionable to operation of a crematorium. In a known light duty prosthesis made of plastic tubing, the residue from the cremated plastic tubes yields a significant quantity of melted plastic presenting a collection and disposal problem for the crematorium. This is a serious concern for the crematorium operator as the melted plastic will “gum up” the floor of the cremation chamber. Removing and cleaning up the material, which may adhere to the floor, can result in flooring being removed with the plastic residue. In any event the extra work necessary to clean up the gummy residue, and possibly replacing flooring, is an expense in time and manpower that is avoided by use of the prosthesis of this invention. Some crematorium operators will avoid the melted plastic problem by removing plastic (PVC pipe and the like) prosthesis limbs before cremation of the cadaver. This of course, is an added processing step in handling the cadaver that is avoided by using the invention disclosed herein.

Returning to FIGS. 5 and 6. In these figures an arm bone prosthesis, generally 34, is shown. This is operationally similar, as far as the telescoping components and the eccentric cam locking device, to the leg bone prosthesis shown in FIGS. 1-4 and is made of consumable materials for ease of disposal after the host cadaver is cremated. In both of the arm bone prosthesis parts, as described below, the eccentric can locking device as described above with respect to the leg bone prosthesis, is used to stabilize the extendable tubes relative to each other.

FIG. 5 shows an unextended arm prosthesis device comprising two major parts. A first part 36 is used as a lower arm prosthesis. This first part component includes a tube 36 of consumable material, in one embodiment the material is cardboard having a moisture resistant coating. A dowel 40, in one embodiment, a wooden dowel, has an outside diameter similar to the inside diameter of the tube 38 so that the tube 38 and the wooden dowel 40 will telescope relative to each other. An eccentric locking device is used in the lower arm in the location generally 24 (the same identification number is used in all the figures for the eccentric cam element). This is the same eccentric cam device that is shown in FIGS. 3 and 4 and used in devices shown in FIGS. 1,2, and 5-8.

A projection 70, such as a nail or staple, may be positioned to project from the dowel 40 within an inch or so of the end of the dowel 40. This projection prevents the tube 36 from sliding too far inboard along the dowel which could allow the cam 24 from being pushed out the end of the tube 36.

A bore is formed in the end of the dowel 40 at the end of the dowel that doesn't have the cam device attached thereto. The bore will accommodate a flexible, bendable connector 56 as described below.

The second part of the arm prosthesis, item 42, representing the upper arm of the recipient cadaver, is comprised of several parts including a tube 44 also made of consumable material, such as, but not limited to, cardboard coated in a moisture resistant coating such as a single ply of plastic wrap formed integrally with the manufacture of the cardboard tube. At one end of the second part 42 of the arm prosthesis generally 34, is a enlarged portion 46 representing or simulating a shoulder. A wooden dowel 48 of the upper arm 42 has an enlarged portion 50 of consumable material. This enlarged portion 50 represents an elbow of the prosthesis generally 34. The wooden dowel 48 is a slidable fit in the tube 44. The end of the wooden dowel 48 is provided with a hole (not shown) drilled therein. This hole in the wooden dowel 48 accommodates a flexible, bendable connector 56. The other end of the connector 56 is positioned in the hole in the end of the dowel 40. The connector 56 may be a short piece, on the order of three inches in length, of electrical wire having a diameter of about five/sixteenths inch. The connector is designed to be bent into a desired position to, for instance, allow the arm of the cadaver into which the arm prosthesis is inserted to be folded to an at rest position without interference between the upper arm 42 portion and the lower arm portion 36. FIG. 6 shows such folded positioning of the arm prosthesis.

An eccentric locking device is also used in the upper arm portion of the arm prosthesis in the location generally 24.

In FIG. 6 both the lower arm section 36 and the upper arm section 42 are extended. The lower arm tube 38 has been slid outwardly on the wooden dowel 40. The upper arm portion 42, the second part of the arm prosthesis, is extended such that the tube 44 of the upper arm is extended along the wooden dowel 48 of the upper arm portion. Proper location of the arm prosthesis will be such that the shoulder portion 46 and the elbow portion 50 approximate the natural locations in the cadaver's arm. The eccentric cam devices, each shown generally as 24, in the upper and the lower arm portions are engaged by rotating the tubes relative to the dowels to secure the tubes and the wooden dowels at the desired lengths.

As can be envisioned, the arm bone prosthesis, once positioned in the cadaver can remain with the cadaver through the cadaver's transportation and final disposition whether it be buried or cremated as discussed above with respect to the leg prosthesis. The arm bone prosthesis is also consumable to a large extent in the cremation process. The cardboard tubes and the wooden dowels are fully consumable. The arm prosthesis is fully consumable except for the eccentric cam blocks and the bendable portion 56, the totality of these several elements is such an insignificant mass of plastic and wire that it poses no problem as far as residue from a cremation operation is concerned. Even in an arm prosthesis of plastic material, as is known in prosthesis of this type, the residue from the cremated plastic tubes yields a significant quantity of melted plastic presenting a collection and disposal problem for the crematorium as addressed above.

FIG. 7 is a view of an unextended upper arm component. It is common in this field to need an upper arm prosthesis only, rather than an arm prosthesis that includes both the upper and lower arm segments. The device shown in FIG. 7 is simply the upper arm portion shown in FIG. 5. It includes the enlarged portion 46 simulating a shoulder, the enlarged portion 50 simulating an elbow carried on the tube 44 and the wooden dowel 48 respectively. The FIG. 7 component will be extendable to the desired length of the upper arm in the recipient cadaver and secured in the extended position using the cam locking element generally 24. There is no need for the connector receiving hole in the end of the dowel in this application.

FIG. 8 is another embodiment of a light duty prosthesis. FIG. 8 shows a spinal column prosthesis generally 58. It is similar to the upper arm prosthesis shown in FIG. 7 in that it includes enlarged portions 60 that will interface with vertebra of the cadaver. A wooden plug, not shown, will be inserted into the end of the tube 62. The wooden plug inserted into the end of the tube 62, which may be of another material, such as but not limited to, plastic or cardboard material, will define an aperture into which a peg or rod 68 is carried. A second rod 66 is inserted in a bore formed in the end of the wooden dowel 64. These rods, 66 and 68, are inserted into the spinal column of the recipient cadaver to locate the spine prosthesis in the recipient cadaver. The spinal column prosthesis, generally 58, operates in the manner disclosed above with respect to the other prosthesis products, that is, it is extended and secured in position using the eccentric cam locking device generally 24.

In summary the leg prosthesis comprises a consumable light duty prosthesis having a tubular element with an inside diameter; a wooden dowel element having an inboard end and having a diameter allowing insertion of the wooden dowel element into the tubular element; and a cam locking device having an eccentric cam element affixed to the inboard end of the wooden dowel element. The cam locking device comprises a cylindrical block having a through bore. The through bore and a pilot hole formed in the end of the wooden dowel are eccentrically offset relative to each other. The cam locking device also includes an axle, which can be a threaded fastener, carried in the through bore of the cylindrical block and extending into the pilot hole of the wooden dowel. The cylindrical block is free to rotate eccentrically on the axle. It is advantageous to have a projection, in one embodiment a longitudinal rib or, in a preferred embodiment, multiple longitudinal ribs, projecting from the surface of the cylindrical block. These ribs will increase the friction between the cam lock block surface and the interior of the cardboard tube, or the like, to decrease the tendency of the cam block to slip inside the tube when the tube and the dowel are rotated relative to each other.

The tube and the wooden dowel are an interference fit when the wooden dowel is positioned in the tube and the wooden dowel is rotated relative to the tube. This restraint system is a significant improvement over the devices known to the inventor as there is no need for the pin-in-hole arrangement used in other devices.

The leg prosthesis includes a hip emulating element that may be fixedly attached to one end of the tube, the end opposite the end of the tube that will accept the wooden dowel and cam locking device, of the tube. The hip emulating element, in one embodiment, is a low density expanded foam element.

A second foam element, again in one embodiment, a low density expanded foam element, simulating a knee joint, is slidably carried on the tube. It will be slidable along the tube to approximate a knee position.

Toward the end of the wooden dowel, at the end not normally inserted into the inside diameter of the tube, an enlarged end element simulating an ankle joint is positioned at the end of the wooden dowel. This ankle joint element may be a section of tubing similar in internal and external dimension to the tube. In a manufacturing situation the tubing used for the tube is simply cut into short pieces and affixed to the end of the wooden dowel to form the simulated ankle joint. Obviously this ankle joint, as well as the simulated hip and knee joints, are not articulatable as articulation is not needed.

It is desirable that the tubes of the various prosthesis products disclosed herein, in one embodiment these being cardboard tubes, are moisture resistant. This can be accomplished by incorporating a sheet of film stock on or over the cardboard tube used for the tubes. Also, the tubes can be sprayed or otherwise coated with a moisture resistant coating.

The single part upper arm prosthesis, shown in FIG. 7, the two part upper and lower arm prosthesis shown in FIGS. 5 and 6, and the spinal column prosthesis shown in FIG. 8 all operate on the same principal as the leg bone prosthesis shown in FIGS. 1-4 and use the cam locking device generally 24.

In addition to the apparatus described above, the inventor contemplates that the method of using the consumable light duty prosthesis as described to replace a removed natural bone can be implemented with the apparatus. Such apparatus for use in performing the method will include a tubular element having a inside diameter; a wooden dowel element having an inboard end and having an outside diameter allowing insertion of the wooden dowel element into the tubular element; and a cam locking device having an eccentric cam element affixed to the inboard end of the wooden dowel element.

This disclosure sets forth an embodiment that satisfies and meets the objects and advantages of the invention set out above. Accordingly, neither the above description of preferred exemplary embodiments, nor the abstract defines or constrains the invention. Rather, the issued claims variously define the invention. Each variation of the invention is limited only by the recited limitations of its respective claim, and equivalents thereof, without limitation by other terms not present in the claim.

In addition, aspects of the invention are particularly pointed out in the claims using terminology that the inventor regards as having its broadest reasonable interpretation; the more specific interpretations of 35 U.S.C. § 112(6) are only intended in those instances where the terms “means” or “steps” are actually recited. The words “comprising,” “including,” and “having” are intended as open-ended terminology, with the same meaning as if the phrase “at least” were appended after each instance thereof.

1. A consumable light duty prosthesis comprising:
a tubular element having a inside diameter;
a dowel element having an inboard end and having an outside diameter allowing insertion of the dowel element into the tubular element;
a cam locking device having an eccentric cam element affixed to the inboard end of the dowel element.

2. The invention set forth in claim 1 wherein the cam locking device comprises a cylindrical block having a through bore, a pilot hole formed in the dowel element, the through bore and the pilot hole eccentrically offset relative to each other, an axle carried in the through bore of the cylindrical block, the axle extending into the pilot hole of the dowel.

3. The invention set forth in claim 2 wherein the axle is a threaded fastener that is threaded into the pilot hole of the dowel.

4. The invention in accordance with claim 3 wherein the cylindrical block is free to rotate on the axle.

5. The invention in accordance with claim 4 wherein the cylindrical block includes a protrusion projecting from the surface of the cylindrical block.

6. The invention in accordance with claim 5 wherein the protrusion comprises a longitudinal rib.

7. The invention in accordance with claim 5 wherein the tube and the dowel element are an interference fit when the dowel element is positioned in the tube and the dowel is rotated relative to the tube.

8. The invention in accordance with claim 7 wherein a hip emulating element is fixedly attached to one end of the tube.

9. This invention in accordance with claim 8 wherein the hip emulating element is a low density expanded foam element.

10. The invention in accordance to claim 7 wherein a second foam element is slidably carried on the tube.

11. The invention in accordance with claim 10 wherein the second foam element further comprises a enlarged end element at the end of the tube.

12. The invention in accordance with claim 7 wherein the tube is moisture resistant.

13. The invention in accordance with claim 5 wherein a plurality of longitudinal ribs are formed on the exterior surface of the cylindrical block.

14. A consumable light duty arm bone prosthesis comprising:
an upper arm portion having a tube having an inside diameter;
the upper arm portion also having a dowel with an outside diameter allowing insertion of the dowel into the tube;
a lower arm portion of the arm bone prosthesis having a tube and a dowel, the dowel carried in the tube of the lower arm portion of the arm bone prosthesis;
a bendable portion connecting the upper arm portion to the lower arm portion;
a bore formed in the upper arm dowel to accommodate the bendable portion;
a bore formed in the lower arm dowel to accommodate the bendable portion, where in the bendable portion is inserted into the bores of the upper arm dowel and the lower arm dowel to connect the upper arm dowel and the lower arm dowel together.

15. The invention in accordance with claim 14 wherein the upper arm portion of the arm bone prosthesis further comprises an enlarged portion simulating an elbow at the end of the dowel.

16. The invention in accordance with claim 15 further comprising an enlarged portion on the tube of the upper arm, the enlarged portion simulating a shoulder.

17. The invention in accordance with claim 14 wherein the tube of the lower arm is provided with a moisture resistant surface.

18. The invention in accordance with claim 17 wherein the tube of the upper arm is provided with a moisture resistant surface.

19. The method of replacing a bone that has been removed from a cadaver leaving a cavity, the method comprising the act of inserting a bone prosthesis in the cavity, the bone prosthesis comprising:
a tubular element having an inside diameter;
a dowel element having an inboard end and having an outside diameter allowing insertion of the dowel element into the tubular element;
a cam locking device having an eccentric cam element affixed to the inboard end of the dowel element.

20. The method as set forth in claim 19 wherein the cam locking device comprises a cylindrical block having a through bore, a pilot hole formed in the dowel element, the through bore and the pilot hole eccentrically offset relative to each other, an axle carried in the through bore of the cylindrical block, the axle extending into the pilot hole of the dowel.
